SEQUENCE LISTING (1 sequence)

SEQ ID NO: 1 - DNA, 579 bp, House Mouse (mus musculus)
atgggcgtgcccgagcgccccaccctgctgctgctgctgagcctgctgctgatccccctgggcctgcccgtgctgtgcgcccccccccgcctgatctgcgacagccgcgtgctggagcgctacatcctggaggccaaggaggccgagaacgtgaccatgggctgcgctgagggcccccgcctgagcgagaacatcaccgtgcccgacaccaaggtgaacttctacgcctggaagcgcatggaggtggaggagcaggccatcgaggtgtggcagggcctgtccctgctgtctgaggccatcctgcaggcccaggccctgctggccaactcctcccagccccccgagaccctgcagctgcacatcgacaaggccatcagcggcctgcgctccctgacctccctgctgcgcgtgctgggcgcccagaaggagctgatgagcccccccgacaccaccccccccgcccccctgcgcaccctgaccgtggacaccttctgcaagctgttccgcgtgtacgccaacttcctgcgcggcaagctgaagctgtacaccggcgaggtgtgccgccgcggcgaccgctga

(19) United States
(12) Patent Application Publication
(10) Pub. No.: US 2005/0004055 A1
(43) Pub. Date: Jan. 6, 2005

(54) Increasing electro-gene transfer of nucleic acid molecules into host tissue

(76) Inventors: Elena Fattori, Rome (IT); Nicola La Monica, Rome (IT); Carmela Mennuni, Rome (IT)

Correspondence Address: MERCK AND CO INC, P O BOX 2000, RAHWAY, NJ, 070650907, US

(21) Appl. No.: 10/495,669

(22) PCT Filed: Nov. 21, 2002
(86) PCT No.: PCT/EP02/13097

Related U.S. Application Data
(60) Provisional application No. 60/333,338, filed on Nov. 26, 2001.

(51) Int. Cl. A61K 48/00; A61K 38/47

(52) U.S. Cl. 514/44; 424/94.61

(57) ABSTRACT

A method of delivering a pharmaceutical agent, such as a nucleic acid molecule, to a vertebrate host is disclosed which comprises combining the synergistic steps of electrical stimulation with administration of a biologically active amount of hyaluronidase. Additionally, formulations which comprise hyaluronidase and a pharmaceutical agent, such as a nucleic acid molecule, are disclosed. The hyaluronidase preparation is preferably administered prior to or simultaneous with the pharmaceutical agent and in conjunction with an applied electrical stimulation, thus affecting increased transfer of the population of a pharmaceutical agent into the target tissue when compared to the affect of electrical stimulation alone. The combination of hyaluronidase administration and electrostimulation results in a substantial increase in the transfer of the pharmaceutical agent, such as a nucleic acid molecule, to the target vertebrate host tissue.

### CROSS-REFERENCE TO RELATED APPLICATIONS

This application claims benefit, under 35 U.S.C. §119(e), to U.S. provisional application 60/333,338 filed Nov. 26, 2001.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to methods of increasing the efficiency of electrical-based transfer of pharmaceutical agents, such as nucleic acid molecules, into a vertebrate host, such as a human or animal host. The methods of the present invention further concern administration of a biologically effective amount of hyaluronidase prior to or simultaneous with administration of the respective pharmaceutical agent and application of an electrical stimulation, thus affecting increased transfer of a population of the pharmaceutical agent into the target tissue when compared to the affect of electrical stimulation alone. Such methodology represents an improved efficiency of transfer and expression of nucleic acid molecules with the target tissue of the respective host. The present invention may also be used in conjunction with other compounds such as proteins and peptides.

Formulations comprising hyaluronidase and a pharmaceutical agent are also disclosed. Such formulations allow for a single administration of these two components in conjunction with an appropriate electrical stimulus.

### BACKGROUND OF THE INVENTION

Studies have shown that applied electrical energy can affect a biological membrane, in that a sufficient application of energy increases the permeability of the membrane and thus allows solutions to diffuse through a membrane or tissue more readily to achieve a desired effect. Generally, electrical or electromagnetic stimulation effects have been explained with reference to one or more of iontophoresis, electrophoresis or electroporation (collectively “electrical stimulation” or “electrostimulation”, or in the context of the transfer of nucleic acid molecules, “electro-gene transfer”, or “EGT”), which are either different forms of electrical stimulation or different ways to interpret the effects of an electromagnetic field. Iontophoresis generally concerns the introduction of an ionized substances through an intact membrane such as the skin, by application of a direct electric current. The current presumably entrains the ions and/or increases ion mobility in the tissue. Electrophoresis concerns the migration of ions in a fluid or gel under influence of an electric field. In electroporation, an electric field (often pulsed) and the associated induced current, induce microscopic pores to form in a membrane, typically a cell membrane. These pores are commonly called “electropores” and the process of forming them is electroporation. A potential application of electroporation is that solutions such as pharmaceutical agents, molecules, ions, and/or water can pass more readily from one side of the membrane to the other through the electrically generated pores. The pores preferably persist temporarily during application of the field. After application of the field, the pores should close or heal within a short period of time. However, the healing time is dependant on the amplitude and duration of the electrical stimulation, and it is possible to damage tissue permanently by application of too high an instantaneous power level and/or too long a duration of stimulation. The damage could be due to formation of untenably large or numerous pores, or resistive heating of the tissue, or both.

Electrically induced pores have been observed and studied to a degree, in vitro, where cells in a solution are substantially independent of one another and are exposed to view. The situation is not readily observed in vivo. If an observation could be made at a particular site and on the microscopic scale that might be most pertinent, it would likely be atypical due to the effects of field and current density variations in the tissue or as induced by the apparatus employed to make the 0.30 observation.

Genetic and immunological therapies are candidates for the electroporation of tissues. In electrical stimulation of tissues, contact and non-contact apparatus are possible. In a contact apparatus, a signal is applied by physically contacting a target tissue site using conductive electrodes attached on opposite sides of the target site. In a non-contact apparatus, an electric or magnetic field can be generated using electrodes or coils that are likewise disposed on opposite sides of the site. In the contact example, the tissue may have a reactive component (capacitance or inductance) and the conductivity of the tissue may change over time due to the effects of the application of energy (e.g., due to heating), but in general the electrical response of the tissue is according to Ohm's law.

Direct plasmid DNA gene transfer is currently the basis of many emerging therapeutic strategies as it avoids the potential problems associated with viral genes and lipid particles (e.g., see van Deutekom et al., 1998; Mol. Med. Today 4: 214-220; Treco and Selden, 1995, Mol. Med. Today 1: 314-321). Skeletal muscle-borne plasmids have been expressed efficiently over months or years in immunocompetent hosts (e.g., see Wolff et al., 1992, Hum. Mol. Genet. 1: 363-369; Davis et al., 1993; Manthorpe et al., 1993, Hum. Gene Ther. 4: 419-431) leading to transgene expression and physiological or therapeutic responses, such as vaccinal and anti-inflammatory response or hematocrit (Hct) increase (e.g., see Davis et al., 1996; Proc. Natl Acad. Sci. USA 93: 7213-7218; Kessler et al, 1996; Proc Natl Acad Sci USA. 93: 14082-14087; Kreiss et al, 1999, J. of Gene Med. 1: 245-250; Levy et al., 1996, Gene Therapy 3: 201-211; Miller et al., 1995, Gene Therapy 2: 736-742; Song et al., 1998; J. Clin. Invest. 101: 2615-2621; Trypathy et al., 1996, PNAS 93: 10876-10880. However, the high individual variability of foreign gene expression, and the low level of therapeutic protein expression, particularly in large animals (see Jiao et al., 1992, Hum. Gene Therapy 3: 21-33) are limiting factors to the use of naked DNA injection for clinical application. Nonetheless, the development of an efficient transfer method for plasmid DNA would be ideal for applications in a variety of diseases.

U.S. Pat. No. 6,110,161, issued Aug. 29, 2000 to Mathiesen et al. (see also, WO 98/43702 and Mathiesen, 1999, Gene Therapy 6: 508-514) disclose in vivo electrostimulation of skeletal muscle within a calculated electric field strength ranging from about 25 V/cm to about 250 V/cm.

WO 99/01158, WO 99/01157 and WO 99/01175 disclose the use of low voltage for a long duration to promote in vivo electrostimulation of naked DNA. An electric field strength or voltage gradient of about 1 V/cm to about 600 V/cm is disclosed, depending upon the target tissue. This encompasses a relatively expansive range from minimal effect to potentially injurious levels. However, even higher voltage gradients have been proposed.

U.S. Pat. No. 5,810,762, U.S. Pat. No. 5,704,908, U.S. Pat. No. 5,702,359, U.S. Pat. No. 5,676,646, U.S. Pat. No. 5,545,130, U.S. Pat. No. 5,507,724, U.S. Pat. No. 5,501,662, U.S. Pat. No. 5,439,440 and U.S. Pat. No. 5,273,525 disclose electroporation/electrostimulation methodology and related apparatus wherein it is suggested that a useful electrical field strength range within the respective tissue is from about 200 V/cm to about 20 KV/cm. U.S. Pat. Nos. 5,968,006 and 5,869,326 further suggest that electric field strengths as low as 100 V/cm are useful for certain in vivo electrostimulation procedures.
Jaroszeski et al. (1999, Advanced Drug Delivery Reviews 35: 131-137) review the present landscape of in vivo electrically mediated gene delivery techniques. The authors emphasize previous success with delivery of chemotherapeutic agents to tumor cells and discuss some of the early results in this field.
Titomirov et al.(1991, Biochem Biophys Acta 1088: 131-134) delivered two plasmid DNA constructs subcutaneously followed by electrical stimulation of skin folds, generating an electric field strength from 400 V/cm to 600 V/cm.
Heller et al. (1996, FEBS Letters 389: 225-228) delivered plasmid DNA expressing two reporter genes to rat liver tissue by generation of high voltage pulses (11.5 KV/cm) rotated through a circular array of electrodes.
Nishi et al. (1996, Cancer Res. 56: 1050-1055) delivered plasmid DNA expressing a reporter gene to rat brain tissue. The authors utilized an electric field strength of approximately 600 V/cm.
Zhang et al. (1996, Biochem. Biophys. Res. Comm. 220: 633-636) delivered plasmid DNA transdermally to mouse skin with 120V pulses to the skin folds wherein the distance between the electrodes was only about 1 mm.
Muramatsu et al. (1997, Biochem. Biophys. Res. Comm. 223:45-49) reported transfection of mouse testis cells with plasmid DNA via 100 V pulses with a 10 mS pulse duration.
Rols et al. (1998, Nature Biotechnology 16(2): 168-171) reported transfection of mouse tumor cells with plasmid DNA by applying voltages from about 300 to 400 V across a 4.2 mm spacing of the electrodes.
Aihara and Miyazaki (1998, Nature Biotechnology 16: 867-870) reported in vivo expression of β-gal in mouse muscle tissue by delivering a square waveform pulse (50 mS duration) at constant voltage (60V) with the distance between the electrodes being 3-5 mm.
Vicat et al. (2000, Human Gene Therapy 11: 909-916) show that high voltage (900 V), short pulse (100_S) electrostimulation protocols result in prolonged expression within targeted cells, in this case mouse muscle cells.
Widera et al (2000, J. Immunology 164: 4635-4640) apply 100 volts over a 5 mm distance with conducting electrodes to deliver hepatitis B surface antigen, HIV gag and env encoding DNA vaccines in vivo to mouse and guinea pigs.
Suzuzki et al. (1998, FEBS Lett. 425: 436-440) apply a voltages of 25, 50 and 100 V to the liver lobe of a rat. The authors found that 8 pulses (50 ms each) of 50 V was optimal for GFP expression.
Goto et al. (2000, Proc. Natl. Acad. Sci. USA. 97: 354-359) show delivery of the “A” fragment of diphtheria toxin and the HSV TK gene to mouse tumors via voltage pulses (with an electric field strength of approximately 66 V/cm) through a circular array of six needle electrodes reduces tumor growth in mice.
Oshima et al. (1998, Gene Therapy 5: 1347-1354) show EGT to rat corneal endothelium.
Favre et al. (2000, Gene Therapy 7: 1417-1420) shows that HYAse enhances adeno-associated virus mediated gene transfer in rat skeletal muscle by increasing viral diffusion in the injected tissue
Fromes et al. (2000, Gene Therapy 6: 683-688) show that a mix of HYAse and collagenase increase adenovirus diffusion in rat myocardium.
Batra et al. (1997, J. Biol. Chem. 272: 11736-11743) show inhibition of retroviral gene transfer to cancer cells by extracellular components of malignant pleural effusion, and neutralization of this inhibition by treatment of effusions with HYAse and chondroitinases.
Dubensky et al. (1984, Proc. Natl. Acad. Sci. USA, 81: 7529-7533) show that injecting polyoma-plasmid recombinant DNA along with HYAse and collagenase leads to more uniform transfection of mouse livers and spleens.

It would be advantageous to identify improved methods of electrical-based transfer of pharmaceutical agents into host tissue which provide for enhanced and long lasting gene expression without any significant tissue alteration. The present invention addresses and meets these needs by disclosing methodology which comprises administration of a biologically effective amount of hyaluronidase in combination with electrical stimulation to increase the gene transfer and expression within host tissue.

### SUMMARY OF THE INVENTION

The present invention relates to a method of delivering a pharmaceutical agent into a tissue of a vertebrate host which comprises the steps of administering a biologically effective amount of hyaluronidase to the tissue of the vertebrate host; administering the pharmaceutical agent proximal to the delivery points of HYAse administration and, applying an electrical stimulus proximal to the site of administration of the hyaluronidase and the pharmaceutical agent. To this end, the methodology of the present invention relates to delivery of a pharmaceutical agent, such as a population of nucleic acid molecules (exemplified herein as DNA plasmid molecules), into the tissue of a vertebrate host, which comprises a) administering a biologically effective amount of hyaluronidase to the tissue of the vertebrate host; b) administering the pharmaceutical agent proximal to the delivery site of hyaluronidase administration in step a); and, c) applying an electrical stimulus proximal to the delivery points of step a) and step b). This methodology results in a substantial increase in delivery, and hence in vivo efficacy, of electrical-based delivery technology.

One aspect of the present invention relates to methods of enhancing the electro-gene transfer (EGT) of nucleic acid molecules into a host vertebrate tissue which comprises administering hyaluronidase (HYAse) in combination with a physiologically acceptable EGT protocol, as described in the above paragraph. The combination of a particular EGT protocol with a HYAse injection results in increased transfer of nucleic acid molecules as compared to application of the respective EGT protocol alone.

The present invention also relates to pharmaceutical formulation which comprises an effective amount of hyaluronidase and the respective pharmaceutical agent. A preferred pharmaceutical agent is an effective concentration of nucleic acid molecules, and most preferably a biologically effective concentration of DNA plasmid molecules.

To this end, the present invention relates to methods of enhancing electro-gene transfer (EGT) of nucleic acids into vertebrate tissue which comprises administering a biologically effective amount of hyaluronidase (HYAse) in combination with an EGT treatment.

To this end, the present invention relates to methods of enhancing EGT of nucleic acids into mammalian tissue which comprises administering a biologically effective amount of hyaluronidase (HYAse) in combination with an EGT treatment.

The present invention further relates to methods of enhancing EGT of nucleic acids into mammalian muscle tissue which comprises administering a biologically effective amount of hyaluronidase (HYAse) in combination with a respective pharmaceutical agent in conjunction with an EGT treatment.

Therefore, a preferred vertebrate target host is a mammal, and an especially preferred target host includes but is not limited to humans and non-human primates, and may also include any non-human mammal of commercial or domestic veterinary importance.

Additionally, while one or more tissue types from the vertebrate host may be targeted for the synergistic EGT/HYAse methodology of the present invention, a preferred tissue type is muscle tissue, which has been shown to be a viable target tissue for various electrostimulation protocols involving gene therapy and/or gene vaccination applications. A preferred mode of administration for either/or of the gene construct and HYAse is by direct needle injection.

A specific embodiment of the present invention relates to the timing of HYAse administration in relation to application of the respective EGT treatment. As shown in FIG. 2, administration of HYAse anywhere from 10 minutes to 4 hours results in an increased efficiency in gene transfer. Therefore, it is preferred that administration of HYAse be prior to or concurrent with application of the EGT treatment. It will be within the purview of the skilled artisan to optimize a specific EGT treatment with a specific time of HYAse administration with a specific target host, knowing that preinjection of HYAse should increase the efficiency of the respective gene transfer protocol. The ability to administer HYAse just prior to or even in conjunction to EGT lends itself to formulations which comprise both HYAse and the respective pharmaceutical agent, such as a nucleic acid molecule. To this end, the present invention relates to a formulation which comprises both HYAse and the respective pharmaceutical agent, such as a nucleic acid molecule, or more preferably, a biologically effective amount of a DNA plasmid construct which expresses the transgene/antigen of interest upon in vivo administration.

A specific embodiment of the present invention thus relates to the increasing the efficacy of electro-gene transfer (EGT) of plasmid DNA into skeletal muscle by preinjecting hyaluronidase (HYAse), which significantly increases the gene transfer efficiency of muscle EGT. Two constructs encoding mouse erythropoietin (PCMV/mEPO) and secreted alkaline phosphatase (pCMV/SeAP) were electro injected intramuscularly in Balb/C mice and rabbits with and without HYAse pretreatment. Preinjection 1 or 4 hr prior to EGT increased EPO gene expression by about 5 fold in mice and maintained higher gene expression than plasmid EGT alone. A similar increment in gene expression was observed upon pretreatment with HYAse and pCMV/mEPO electroinjection in rabbit tibialis muscle. The increment of gene expression in rabbits reached 17 fold upon injection of plasmid pCMV/SeAP.

It is an object of the present invention to provide for an enhancement of EGT-based protocols for, in vivo gene transfer into vertebrate tissues wherein hyaluronidase (HYAse) is administered in combination with a respective pharmaceutical agent and an effective EGT protocol. The combination of a particular EGT protocol with a HYAse injection (preferably prior to, and possibly at or near the time of electrostimulation of tissue surrounding the area of nucleic acid delivery), thus resulting in increased efficiency of gene transfer, expression and/or immunogenicity of a respective gene construct as compared to application of the respective EGT protocol alone.

It is also an object of the present invention to provide for formulations which comprise both HYAse and a respective pharmaceutical agent, such as a population of nucleic acid molecules which, upon in vivo administration, result in expression of a respective transgene(s)/antigen(s).

As used herein, “p.i.” is an abbreviation for—post injection—.

As used herein, “EGT.” is an abbreviation for—electro-gene transfer—, which is used interchangeably with the terms “electrostimulation” and “electrical stimulation.”

As used herein, “HYAse” is an abbreviation for—hyaluronidase—.

### BRIEF DESCRIPTION OF THE FIGURES

FIG. 1A-B shows dependence of plasmid pCMV/mEPO expression on the concentration of HYAse preinjected in Balb/c mice. A. Serum EPO levels. B. Hematocrit levels. Groups of 4 mice were injected in quadriceps muscle with different doses of HYAse 4 hr prior to plasmid EGT. Blood samples were collected 10 days p.i. and compared to EGT alone and saline controls. Data are the mean ±SD of hematocrit and serum EPO. *Significantly different from EGT alone.

FIG. 2 shows the effect of HYAse preinjection time on plasmid EGT enhancement. Groups of 4 Balb/c mice were preinjected with 36 U of HYAse at different times prior to plasmid EGT. Blood samples were collected 7 days p.i. and compared to EGT alone and saline controls. Data are the mean ±SD serum EPO.* Significantly different from EGT alone.

FIG. 3A-C shows the long term effect of HYAse injection on EPO expression. Groups of 4 Balb/c mice were injected with 36 U of HYAse 1 hr prior to plasmid EGT. The serum EPO levels of animals injected with different DNA doses were compared at (A) 7, (B) 56, and (C) 120 days p.i. Data are the mean ±SD serum EPO. *Significantly different from EGT alone.

FIG. 4A-C shows histological analysis of HYAse injected mouse quadriceps. Mice quadriceps were injected with 36 U of HYAse and tissues were analyzed (A) 3, (B) 7, and (C) 30 days p.i.

FIG. 5A-B shows the effect of HYAse injection on plasmid EGT in rabbits. 180 U of HYAse were injected in the tibilias muscle 40 min. prior to plasmid EGT. Rabbits were injected with either 200 μg of plasmid pCMV/mEPOopt or with 200 μg of plasmid pCMV/SeAP. Blood samples were collected 4 days p.i. and serum EPO (A) and SeAP (B) levels were measured and compared to those detected in animals treated with plasmid EGT alone. Data are the mean ±SD as measured in four rabbits. *Significantly different from EGT alone.

FIG. 6 shows expression of β-galactosidase after plasmid pCMV/β-gal/NLS EGT with or without HYAse preinjection. 360 U of HYAse were injected in rabbit tibialis anterior muscle 40 min prior to plasmid EGT. Muscles were collected 4 days p.i. and treated as described in materials and methods section. The left muscle represents tibialis anterior from rabbits pretreated with HYAse, the right muscle represents rabbits electroinjected with plasmid DNA alone.

### DETAILED DESCRIPTION OF THE INVENTION

The present invention relates to methods of enhancing electrostimulation of a pharmaceutical agent, including but not limited to nucleic acid-based formulations, into vertebrate tissue which comprises administering a biologically effective amount of hyaluronidase (HYAse) in combination with an electro-gene transfer treatment.

In other words, the present invention improves upon previous techniques which enhance delivery of the pharmaceutical agent by applying electrostimulation to points proximal to the site of injection (thus, electro-gene transfer, or “EGT”). In the present invention, application of hyaluronidase to (a) the area proximal to the site of administration of the pharmaceutical agent and region of electrostimulation, or (b) simultaneous delivery of hyaluronidase and pharmaceutical agent (preferably in a single formulation or composition) in conjunction with application of electrostimulation, results in an increased transfer of the pharmaceutical agent (exemplified herein with DNA plasmid constructions) as compared to application of the respective electrostimulation protocol alone.

As noted herein, the present invention relates to a method of delivering a pharmaceutical agent into a tissue of a vertebrate host which comprises the steps of administering a biologically effective amount of hyaluronidase to the tissue of the vertebrate host; administering the pharmaceutical agent proximal to the delivery points of HYAse administration and, applying an electrical stimulus proximal to the site of administration of the hyaluronidase and the pharmaceutical agent. To this end, the methodology of the present invention relates to delivery of a pharmaceutical agent, such as a population of nucleic acid molecules (exemplified herein as DNA plasmid molecules), into the tissue of a vertebrate host, which comprises a) administering a biologically effective amount of hyaluronidase to the tissue of the vertebrate host; b) administering the pharmaceutical agent proximal to the delivery site of hyaluronidase administration in step a); and, c) applying an electrical stimulus proximal to the delivery points of step a) and step b). This methodology results in a substantial increase in delivery, and hence in vivo efficacy, of electrical-based delivery technology. It is shown herein that administration of HYAse may occur hours or minutes prior to electrical stimulation. Therefore, the methodology of the present invention logically covers a time spectrum regarding HYAse administration from more than four hours up to minutes, or even in conjunction with electrostimulation of the target tissue. To this end, one aspect of the invention further relates to methodology disclosed herein whereby HYAse and the pharmaceutical agent (such as a DNA plasmid construct) are formulated together in order to allow for a present a single injection at the target site.

To this end, given the exemplification that administration of HYAse may occur hours or minutes prior to electrical stimulation, a preferred aspect of this invention is a pharmaceutical formulation or composition which comprises both an effective amount of hyaluronidase and the respective pharmaceutical agent. A preferred pharmaceutical agent is an effective concentration of nucleic acid molecules, and most preferably a biologically effective concentration of DNA plasmid molecules. Such a formulation will be especially useful for dual administration of HYAse and the pharmaceutical agent in a scenario whereby a only a single injection is required in conjunction with EGT for transfer and expression of nucleic acid-based vehicles.

The present invention therefore relates to formulations and methods of enhancing EGT of nucleic acids into mammalian tissue which comprises administering a biologically effective amount of hyaluronidase (HYAse) in combination with administration of a pharmaceutical agent an EGT treatment. As noted above, a preferred formulation may be a formulation which comprises both HYAse and a pharmaceutical agent, such as an effective amounts of DNA plasmid molecules expressing a transgene of interest.

The present invention further relates to methods of enhancing EGT of nucleic acids into mammalian muscle tissue which comprises administering a biologically effective amount of hyaluronidase (HYAse) in combination with an EGT treatment.

Therefore, a preferred vertebrate target host is a mammal, and an especially preferred target host includes but is not limited to humans and non-human primates, and may also include any non-human mammal of commercial or domestic veterinary importance.

Additionally, while one or more tissue types from the vertebrate host may be targeted for the synergistic EGT/HYAse methodology of the present invention, a preferred tissue type is muscle tissue, which has been shown to be a viable target tissue for various electrostimulation protocols involving gene therapy and/or gene vaccination applications.

Hyaluronidase is utilized in clinical applications. Therefore, the combination of HYAse administration and muscle EGT constitutes an efficient manner in which to enhance to delivery and expression of gene therapy and/or genetic vaccination constructions in order to achieve greater therapeutic and/or prophylactic levels of gene expression within the target host. A particularly preferred application within the field of DNA vaccine technology is the delivery of a DNA vaccine which encodes one or more HIV antigens, including but not limited to HIV Gag, HIV Pol and/or HIV Nef. Formulations which comprise both HYAse and one of the DNA vaccines listed herein comprise one preferred aspect of the present invention. The delivery to and expression from muscle tissue may be enhanced by combining the application of an EGT treatment and administration of a biologically effective amount of HYAse, providing for improved enhanced cellular-mediated immune responses upon host administration. An effect of the improved delivery, expression and/or immunogencity of such DNA vaccines may be a lower transmission rate to previously uninfected individuals (i.e., prophylactic applications) and/or reduction in the levels of the viral loads within an infected individual (i.e., therapeutic applications), so as to prolong the asymptomatic phase of HIV-1 infection. Therefore, the essence of the present invention relates to methodology which provides for an increase in the level of gene expression and/or immune response subsequent to delivery of a respective gene therapy or gene vaccination construction to the muscle tissue of a vertebrate host. A series of preferred hosts include a mammalian host including but not limited to humans and non-human primates, and also include any non-human mammal of commercial or domestic veterinary importance. The methodology, and concomitant formulations which comprise HYAse and the respective pharmaceutical agent, may be applicable to any gene therapy target that relies on the expression of a secreted protein that can exert its biological effect systemically. Examples include but are not limited to gene therapy targets such as EPO, Factor VIII, Factor IX, Growth hormone, various cytokines, and interferon.

It will be understood that any known methodology relating to EGT may be utilized in combination with HYAse to promote increased efficiency of that particular gene transfer methodology within the target host. Briefly, it is known that applied electrical stimulation can affect biological tissues. Applied electrical fields can affect a rate of diffusion through tissues by advection, or may vary the extent to which fluids diffuse into certain parts of the tissues. For example, electrical stimulation can increase permeability of a membrane when it is desired to infuse tissue with a substance through the membrane, and the rate of diffusion is at least partly a function of permeability. Certain electrical or electromagnetic stimulation effects have been explained with reference to iontophoresis, electrophoresis and electroporation. These terms involve different forms electrical effects. They may be considered different ways to interpret the results that are caused by a given electrical potential, current or electromagnetic field. Depending on amplitude, polarity, frequency, spatial geometry and other parameters, a given field may produce a combination of such effects.

Iontophoresis and electrophoresis generally concern applying a direct current electric field in order to drive migration of positive and negative ions by electrostatic attraction and repulsion toward and away from an anode and cathode. Electric fields also tend to increase the mobility of the ions generally. Iontophoresis typically involves causing polar ions in a solution to migrate through an intact membrane such as the skin. Electrophoresis concerns the migration of ions in a fluid or gel under the influence of a polar electric field (i.e., a field with at least a direct current component).

Electroporation often involves a relatively higher power electric field, often applied briefly or pulsed. A field applied at sufficient amplitude and/or for a sufficient duration can induce microscopic pores to form in a membrane. The pores are commonly called “electropores” and the process of forming them is called electroporation. Depending on the power and duration of the energy applied to a membrane, the pores may be larger or smaller and may persist for a longer or shorter time. Preferably the pores persist temporarily, such as only during application of the field, and close or heal quickly. In this disclosure the term “electo-gene transfer” (EGT), “electrical stimulation” and/or “electrostimulation”, used interchangeably herein, is not limited to any one or any particular combination of iontophoresis, electroporation, electrophoresis or any other electrical effects. The terms as used herein are intended to encompass any such effects. A given electrical stimulation could have results that fall into more than one class, or possibly could be stronger in one or another due to the amplitude, polarity, spatial geometry and/or timing involved. For example, a given direct current or low frequency field could conceivably have sufficient amplitude to induce pore formation (electroporation) while also causing electrostatically driven ion migration through a membrane (iontophoresis) and accumulated migration with time (electrophoresis). Typically, however, electroporation involves higher electric field amplitudes than the other effects, and typically application at such amplitudes is brief or intermittent or is pulsed at a duty cycle that is sufficiently low to prevent unacceptable tissue damage.

The application of an electromagnetic field to tissue is complicated by the fact that tissue is not homogeneous, isotropic or otherwise regular from an electromagnetic perspective. An applied field and an induced current can become concentrated by variations in the material properties of the tissue, including but not limited to the magnetic permeability and resistivity of tissues on a microscopic scale, and on a more macroscopic scale, by the anatomical structure and organization of tissues.

Electrically induced pores have been observed and studied to a degree, in vitro, where cells in a solution are substantially independent of one another and are exposed to view. It is difficult or impossible to observe the effects at a particular site in vivo. For example, obtaining access to tissue in vivo, such as sectioning the tissue to expose a site to view, tends to disturb the tissue in ways that alter the local amplitude, orientation or other aspects of the applied electrical energy. Thus it is difficult to make a meaningful in vivo observation of electrical stimulation parameters and effects.

Genetic and immunological therapies are candidates for electrical stimulation of tissues. Inasmuch as electrical stimulation tends to involve movement of ions and the opening of pores in tissues, it is plausible to apply a medicinal or other composition to a tissue site and to use electrical stimulation to move ions or molecules of the composition into positions, perhaps through pores in tissue membranes, where a desired effect is achieved or enhanced. Diffusion from thermal effects (Brownian motion) could drive diffusion through electroporated tissue membranes into an internal volume. Electrostatic or other electromagnetic effects could drive diffusion of ions through biological structures, or at least increase the motion of affected molecules (e.g., assuming an alternating polarity field), and thus affect particular reactions in order to achieve or to induce a therapeutic effect.

The limited gene transfer efficacy characteristic of plasmid DNA injection has been ascribed, at least in part, to the presence of abundant connective tissue, particularly in large animals, that may prevent appropriate contact between the injected DNA and the muscle fiber. This hypothesis is consistent with the idea that the extracellular matrix may play an important role in protecting muscle fibers against penetration by exogenous molecules, bacteria, or virus. Although electrostimulation of muscle tissue may enhance DNA transfer by increasing membrane permeability of muscle fibers, it is probable that the structural characteristic of the extracellular matrix may influence the gene transfer efficiency of the injected DNA across the treated muscle. Thus, enzymatic permeabilization of the extracellular matrix could create pores large enough to allow the productive interaction between the injected DNA and the muscle fiber. It is disclosed herein the effect on muscle EGT of extracellular matrix disruption by an enzymatic treatment (i.d., HYAse treatment) on muscle gene transfer and expression. HYAse hydrolyzes hyaluronic acid which is a ubiquitary constituent of the extracellular matrix. Treatment of mouse and rabbit skeletal muscle with HYAse prior to DNA injection and electrical stimulation results in enhanced and long lasting gene expression without any significant tissue alteration. To this end, the present invention relates to the improvement of electrostimulation methodology by administering a biologically effective amount of hyaluronidase (HYAse). The administration of HYAse will increase the transfer of the gene therapy and/or gene vaccination construction as compared to the respective electrostimulation parameters without administration of HYAse. In other words, HYAse provides for a synergistic increase in gene transfer when utilized in conjunction with electrostimulation methodology. As noted above, any such electrostimulation-based methodology contemplated by the skilled artisan to improve gene transfer may be utilized in conjunction with administration of HYAse to the target host. Variations in EGT parameters may be utilized in practicing the present invention, including but not necessarily limited to varying voltage, the duration of pulse, the rotation of the electric field, the number of pulses, their frequencies, the interval between pulses, as well as the timing of administration of the pharmaceutical agent and electrostimulation. Examples of such techniques include but are not limited to the following: U.S. Pat. No. 6,110,161 (see also Mathiesen, 1999, Gene Therapy 6: 508-514) which discloses in vivo electrical stimulation of skeletal muscle within a calculated electric field strength ranging from about 25 V/cm to about 250 V/cm; PCT International publications WO 99/01158, WO 99/01157 and WO 99/01175, which disclosed the use of low voltage for a long duration to promote in vivo electrical stimulation of naked DNA, with an electric field strength or voltage gradient of about 1 V/cm to about 600 V/cm is disclosed; U.S. Pat. No. 5,810,762, U.S. Pat. No. 5,704,908, U.S. Pat. No. 5,702,359, U.S. Pat. No. 5,676,646, U.S. Pat. No. 5,545,130, U.S. Pat. No. 5,507,724, U.S. Pat. No. 5,501,662, U.S. Pat. No. 5,439,440 and U.S. Pat. No. 5,273,525 which disclose electroporation/electrostimulation methodology and related apparatus wherein it is suggested that a useful electrical field strength range within the respective tissue is from about 200 V/cm to about 20 KV/cm, while U.S. Pat. Nos. 5,968,006 and 5,869,326 further suggest that electric field strengths as low as 100 V/cm are useful for certain in vivo electrostimulation procedures. Additional studies (with varying parameters, as discussed in the Background of the Invention) can be found, for example, in Titomirov et al.(1991, Biochem Biophys Acta 1088; 131-134), Heller et al. (1996, FEBS Letters 389: 225-228), Nishi et al. (1996, Cancer Res. 56: 1050-1055), Zhang et al. (1996, Biochem. Biophys. Res. Comm. 220: 633-636), Muramatsu et al. (1997, Biochem. Biophys. Res. Comm. 223: 45-49), Rols et al. (1998, Nature Biotechnology 16(2): 168-171), Aihara and Miyazaki (1998, Nature Biotechnology 16: 867-870), Vicat et al. (2000, Human Gene Therapy 11: 909-916), Widera et al (2000, J. Immunology 164: 4635-4640), Suzuzki et al. (1998, FEBS Lett. 425: 436-440), Goto et al. (2000, Proc. Natl. Acad. Sci. USA. 97: 354-359), Oshima et al. (1998, Gene Therapy 5; 1347-1354), Rizzuto et al. (1999, Proc. Natl. Acad. Sci. USA 96: 6417-6422), Mir et al., (1998, C. R. Acad. Sci. Paris (Life Science) 321: 893-899), Mir et al, (1999; Proc. Nat. Acad. Sci. USA. 96: 4262-4267); Maruyama et al. (2000, Human-Gene Therapy 11: 429-437) and Draghia-Akli et al. (1999, Nature Biotechnology 17: 1179-1183). The following patent and non-patent publications are hereby incorporated by reference so far as they pertain to known methodology for promoting gene transfer and expression thorough electrostimulation of respective target tissue.

It is shown herein that preinjecting hyaluronidase (HYAse) significantly increases the gene transfer efficiency of muscle EGT. Two constructs encoding mouse erythropoietin (pCMV/mEPO) and secreted alkaline phosphatase (pCMV/SeAP) were electro injected intramuscularly in Balb/C mice and rabbits with and without HYAse pretreatment. Preinjection 1 or 4 hr prior to EGT increased EPO gene expression by about 5 fold in mice and maintained higher gene expression than plasmid EGT alone. A similar increment in gene expression was observed upon pretreatment with HYAse and pCMV/mEPO electroinjection in rabbit tibialis muscle. The increment of gene expression in rabbits reached 17 fold upon injection of plasmid pCMV/SeAP. Injection of a plasmid encoding β-galactosidase (pCMV/βgal/NLS) and subsequent X-gal staining indicated that HYAse increased the tissue area involved in gene expression. No irreversible tissue damage was observed upon histology analysis of treated mouse quadriceps.

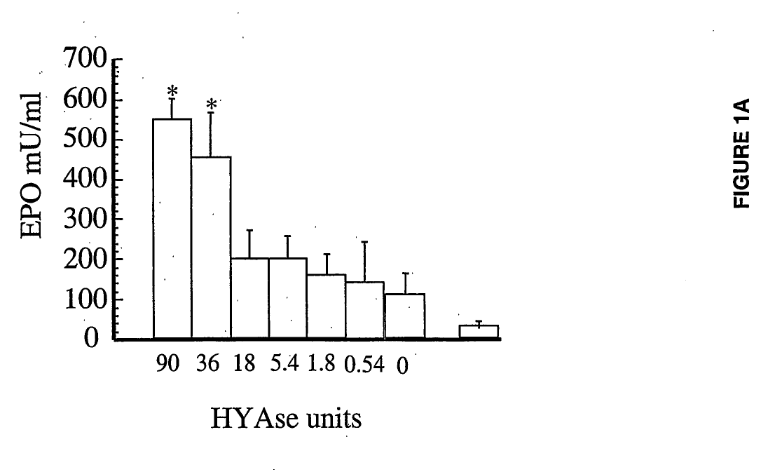

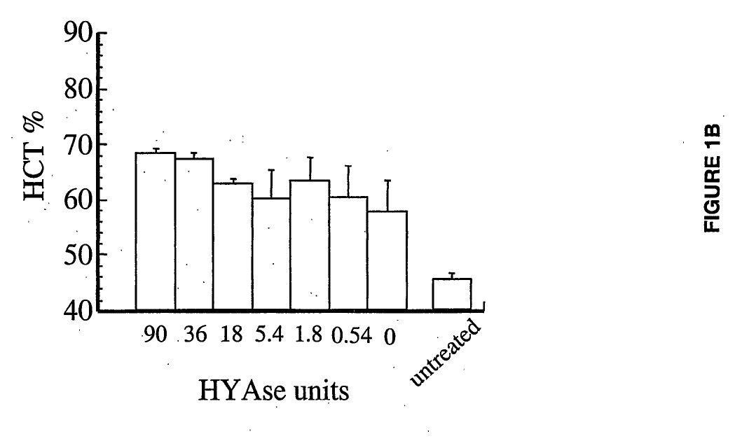

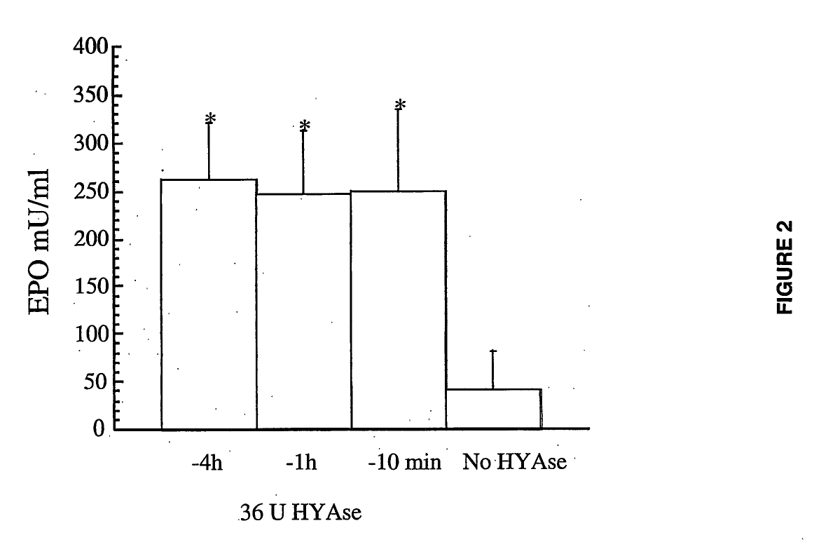

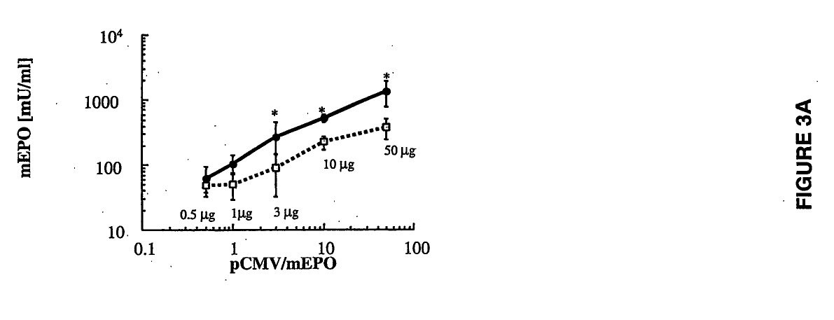

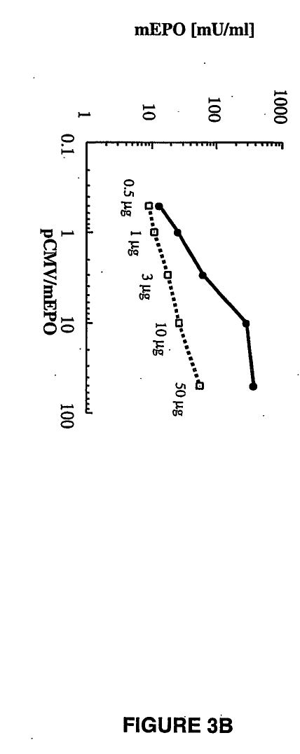

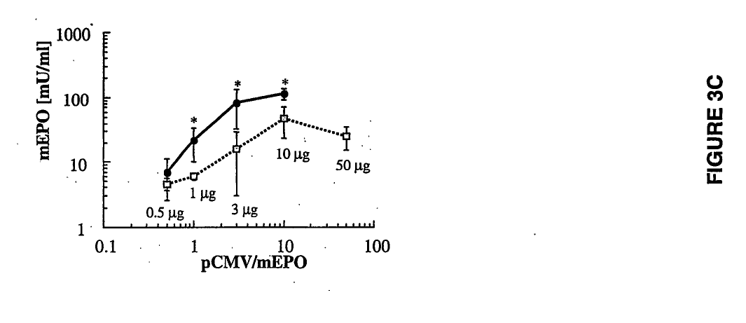

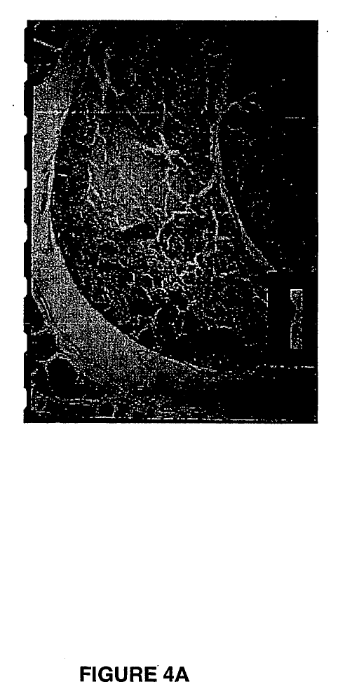

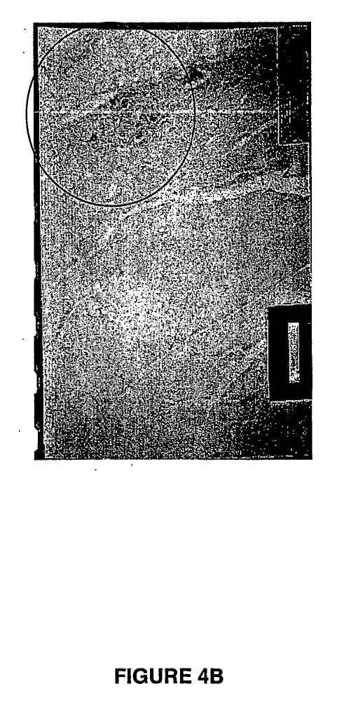

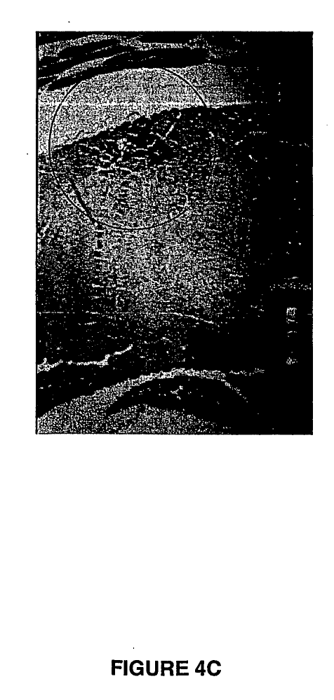

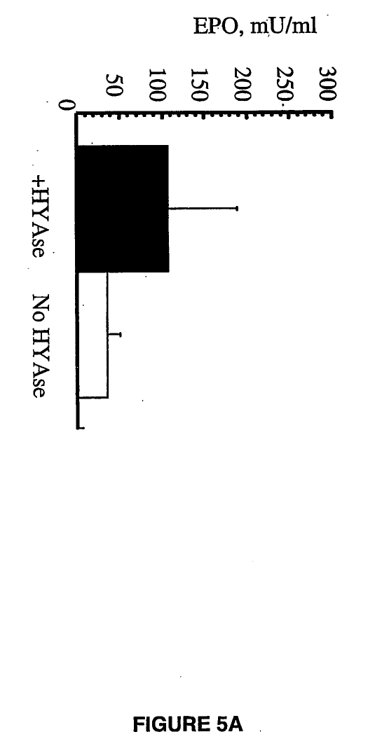

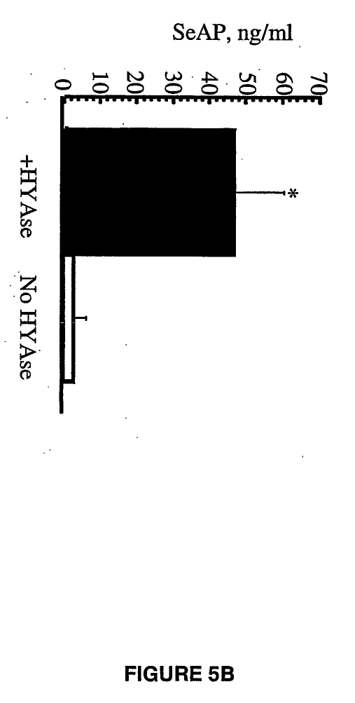

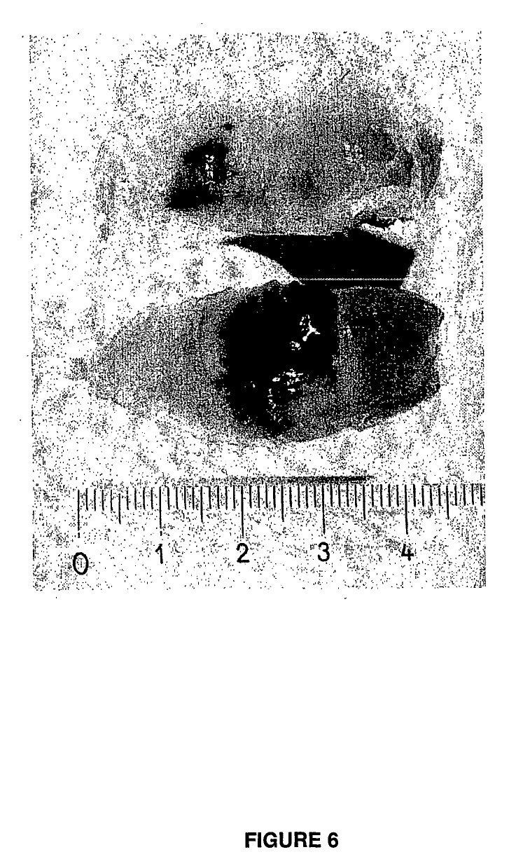

Therefore, the present invention relates to, as exemplified herein, the enhancement of transduction efficiency of EGT by injection of HYAse. It is shown herein that a pretreatment with HYAse has long lasting effects and increases gene expression by three to ten fold. This observation has important implications for the development of a gene transfer protocol suitable for therapeutic applications. Hyaluronidase catalyzes the hydrolysis of the β(14) linkage of hyaluronic acid, leading to its depolymerization and causing a temporary decrease in viscosity in the extracellular ground substance of the connective tissue. It is shown herein (FIG. 1A-B) that HYAse increases transduction efficiency in a dose-dependent manner and is consistent with the mode of action of the enzyme (FIG. 2). Histology experiments with pCMV/NLS/β-gal indicate that HYAse significantly enhances the tissue area involved in gene expression. (FIG. 6). This may be due to an increase of DNA distribution throughout the tissue, thus leading to an augmented bioavailability of plasmid DNA. HYAse treatment may also contribute to releasing plasmid DNA from interactions with components of cellular matrix that may interfere with DNA entry upon electrical stimulation. HYAse administration without muscle ES does not appear to increase gene transfer efficiency, even after injection of 100 μg of pCMV/mEPO. The data disclosed herein shows that HYAse does not directly influence cellular uptake of plasmid DNA but is dependent on muscle electrostimulation to exert its effect on gene expression. These observations distinguish the use of HYAse from sodium phosphate, recently reported as enhancing gene expression in muscle by inhibiting DNA degradation (Hartikka et al., 2000, Gene Therapy 7: 1171-1182.), as well as non-ionic carriers such as polyvinyl pyrrolidone and SP1017 (Lemieux et al., 2000, Gene Therapy 7: 986-991; Mumper et al., 1996, Pharm. Res. 13: 114-121; Alakhov et al., 1995; Bioconj. Chem. 7: 209-216; Batrakova et al., 1996; Br J Cancer 74: 1545-1552). The enhanced gene expression associated to HYAse treatment does not influence the overall stability of injected DNA as shown by the progressive decline in EPO expression that is observed in all injected animals (FIG. 3A-C). Although it is likely that an enhanced DNA transfer across the treated muscle will guarantee a prolonged gene expression, this observation suggests that factors such as DNA stability and promoter attenuation are to be considered important determinants of the efficacy of in vivo gene transfer. Hyaluronidase is currently utilized for clinical applications for such uses as the facilitation of hypodermoclysis, the readsorption of edemas and in the formulation of local anesthetics. Thus, it is not surprising that histology analysis did not reveal significant or permanent alterations of the injected tissues (FIG. 4 A-C), and that animals injected with HYAse did not show any sign of discomfort. This is in contrast with risks of extensive muscle damage associated with the use of potent muscle regenerating agents such as cardiotoxin and bupivacain. A 17-fold increase in expression was observed in rabbits upon injection of plasmid pCMV/SeAP, whereas EPO expression was augmented 3 fold (FIG. 5A-B). The reasons for the differences in enhancement of gene expression between EPO and SeAP may reside in the sensitivity of the detection assays as well as on the stability of the expressed proteins. Alternatively, this difference may reflect a varying efficiency of secretion of EPO and SeAP from skeletal muscle (e.g., see Kreiss et al., 1999, J. of Gene Med. 1: 245-250). Nonetheless, the significant increase in gene expression observed upon HYAse injection in rabbits indicates that the use of this enzyme could guarantee increased gene transfer efficiency in large animals. This conclusion is particularly relevant for the more broad application of muscle EGT for human therapy, which will probably require a small number of injections and a minimal amount of injected DNA along with a sustained expression at therapeutic levels.

The nucleic acid molecules for use in the EGT/HYAse methodology of the present invention may be formulated in any pharmaceutically effective formulation for host administration; As noted throughout this disclosure, a preferred formulation is a formulation which comprises both HYAse and the respective pharmaceutical agent in biologically effective concentrations. Any such formulation may be, for example, in a saline solution such as phosphate buffered saline (PBS). It will be useful to utilize pharmaceutically acceptable formulations which also provide long-term stability of the nucleic acid molecules, such as a DNA plasmid construction. During storage as a pharmaceutical entity, DNA plasmid molecules undergo a physiochemical change in which the supercoiled plasmid converts to the open circular and linear form. A variety of storage conditions (low pH, high temperature, low ionic strength) can accelerate this process. Therefore, the removal and/or chelation of trace metal ions (with succinic or malic acid, or with chelators containing multiple phosphate ligands) from the DNA plasmid solution, from the formulation buffers or from the vials and closures, stabilizes the DNA plasmid from this degradation pathway during storage. In addition, inclusion of non-reducing free radical scavengers, such as ethanol or glycerol, are useful to prevent damage of the DNA plasmid from free radical production that may still occur, even in apparently demetalated solutions. Furthermore, the buffer type, pH, salt concentration, light exposure, as well as the type of sterilization process used to prepare the vials, may be controlled in the formulation to optimize the stability of the DNA molecule. Therefore, formulations that will provide the highest stability of the nucleic acid molecule such as a DNA plasmid vector will be one that includes a demetalated solution containing a buffer (phosphate or bicarbonate) with a pH in the range of 7-8, a salt (NaCl, KCl or LiCl) in the range of 100-200 mM, a metal ion chelator (e.g., EDTA, diethylenetriaminepenta-acetic acid (DTPA), malate, inositol hexaphosphate, tripolyphosphate or polyphosphoric acid), a non-reducing free radical scavenger (e.g. ethanol, glycerol, methionine or dimethyl sulfoxide) and the highest appropriate DNA concentration in a sterile glass vial, packaged to protect the highly purified, nuclease free DNA from light. A particularly preferred formulation which will enhance long term stability of the DNA vector vaccines of the present invention would comprise a Tris-HCl buffer at a pH from about 8.0 to about 9.0; ethanol or glycerol at about 3% w/v; EDTA or DTPA in a concentration range up to about 5 mM; and NaCl at a concentration from about 50 mM to about 500 mM. The use of such stabilized DNA vector vaccines and various alternatives to this preferred formulation range is described in detail in PCT International Application No. PCT/US97/06655 and PCT International Publication No. WO 97/40839, both of which are hereby incorporated by reference.

The nucleic acid molecules described herein may also be formulated with an adjuvant or adjuvants which may increase immunogenicity of the gene therapy or gene vaccination vehicle. A number of these adjuvants are known in the art and are available for use in a DNA vaccine, including but not limited to particle bombardment using DNA-coated gold beads, co-administration of DNA vaccines with plasmid DNA expressing cytokines, chemokines, or costimulatory molecules, formulation of DNA with cationic lipids or with experimental adjuvants such as saponin, monophosphoryl lipid A or other compounds which increase the efficacy of a particular gene therapy or gene vaccination construction. Another adjuvant for use in conjunction with the methodology disclosed herein are one or more forms of an aluminum phosphate-based adjuvant wherein the aluminum phosphate-based adjuvant possesses a molar PO₄/Al ratio of approximately 0.9. An additional mineral-based adjuvant may be generated from one or more forms of a calcium phosphate. These mineral-based adjuvants are particularly useful in increasing cellular and humoral responses to DNA vaccination. These mineral-based compounds for use as DNA vaccines adjuvants are disclosed in PCT International Application No. PCT/US98/02414, PCT International Publication No. WO 98/35562, which is hereby incorporated by reference. Another preferred adjuvant is a non-ionic block copolymer which shows adjuvant activity with DNA vaccines. The basic structure comprises blocks of polyoxyethylene (POE) and polyoxypropylene (POP) such as a POE-POP-POE block copolymer. Newman et al. (1998, Critical Reviews in Therapeutic Drug Carrier Systems 15(2): 89-142) review a class of non-ionic block copolymers which show adjuvant activity. The basic structure comprises blocks of polyoxyethylene (POE) and polyoxypropylene (POP) such as a POE-POP-POE block copolymer. Newman et al. id., disclose that certain POE-POP-POE block copolymers may be useful as adjuvants to an influenza protein-based vaccine, namely higher molecular weight POE-POP-POE block copolymers containing a central POP block having a molecular weight of over about 9000 daltons to about 20,000 daltons and flanking POE blocks which comprise up to about 20% of the total molecular weight of the copolymer (see also U.S. Reissue Patent No. 36,665, U.S. Pat. No. 5,567,859, U.S. Pat. No. 5,691,387, U.S. Pat. No. 5,696,298 and U.S. Pat. No. 5,990,241, all issued to Emanuele, et al., regarding these POE-POP-POE block copolymers). WO 96/04932 further discloses higher molecular weight POE/POP block copolymers which have surfactant characteristics and show biological efficacy as vaccine adjuvants. The above cited references within this paragraph are hereby incorporated by reference in their entirety. It is therefore within the purview of the skilled artisan to utilize available adjuvants which may increase the immune response of the polynucleotide vaccines of the present invention in comparison to administration of a non-adjuvanted polynucleotide vaccine.

The EGT/HYAse methodology of the present invention may call for the administration of either/or of the nucleic acid construction and HYAse by any means known in the art, such as enteral and parenteral routes. The preferred route of administration is intramuscular. Additional routes included but are not limited to subcutaneous administration, intraperitoneal injection, intravenous injection, inhalation or intranasal delivery, oral delivery, sublingual administration, transdermal administration, transcutaneous administration, percutaneous administration or any form of particle bombardment, such as a biolistic device such as a “gene gun” or by any available needle-free injection device. The preferred method of delivery of the nucleic acid construction and HYAse intramuscular injection via needle in conjunction with a respective EGT protocol. Additional methods of delivery include but are not necessarily limited to subcutaneous administration and needle-free injection. A particular mode of administration is the use of a sort of ointment, as noted above.

The amount of expressible DNA to be introduced to a host recipient will depend on the strength of the transcriptional and translational promoters used in the DNA construct, and on the level of expressed protein required to treat the disease or disorder, or on the immunogenicity of the expressed gene product. In general, an effective dose of about 1 μg to greater than about 20 mg, and preferably in doses from about 1 mg to about 5 mg is administered directly into muscle tissue. As noted above, subcutaneous injection, intradermal introduction, impression through the skin, and other modes of administration such as intraperitoneal, intravenous, inhalation and oral delivery are also contemplated. It is also contemplated that booster applications may be utilized, which will again be construct and disease specific, so as to optimize the effectiveness of the gene therapy or gene vaccination application.

The following examples are provided to illustrate the present invention without, however, limiting the same hereto.

### EXAMPLE 1

Plasmid preparation—Constructs pCMV/mEPO, pCMV/β-gal/NLS, and pCMV/SeAP were constructed as follows: The complete mouse EPO (mEPO) coding region, including 40 bp of the 5′ untranslated region (Shoemaker and Mitsock, 1986, Mol. Cell Biol. 6: 849-858) was assembled from synthetic oligonucleotides as described (Stemmer et al., 1995, Gene 164: 49-53), with minor modifications. Briefly, 15 oligos, 60 nt in length, were used after gel purification: 8 oligos covered part of the sequence of the plus strand, whereas 7 oligos covered part of the sequence of the minus strand, and the oligos were configured in such a way that, upon assembly, they overlapped with regions of complementarity of 20 nt. Two SacI sites and two PstI sites present in the mEPO coding sequence were eliminated without altering the encoded protein sequence and, at the same time, optimizing the codon usage. Gene assembly was performed as described (Stemmer et al., 1995, id.), and the entire coding region was verified by dideoxy sequencing. Plasmid pCMV/mEPO was constructed by inserting the mEPO coding sequence as a EcoRI-BamHI 0.6 Kb fragment into pViJnsB (Montgomery et al. 1993, DNA Cell. Biol. 12: 777-783) containing the CMV immediate/early region promoter and enhancer with intron A followed by the BGH polyadenylation signal. To construct pViJ/β-gal/nls a 3.5 Kb BamHI β-gal/nls encoding fragment was excised from pGM48β-gal (Wiznerowicz et al., 1997) and cloned in the BglII restriction site of pViJnsB (Montgomery et al., id). Plasmid pViJ/SEAP was constructed by inserting the coding sequence of secreted alkaline phosphatase in the BglII restriction site of pViJnsB (Montgomery et al., id.).

Plasmid pCMV/mEPOopt carries the mouse EPO cDNA codon-optimized to mammals. Briefly, the EPO cDNA coding sequence was modified such that the native codons were substituted with codons frequently found in highly expressed human genes. Th optimized sequence of mouse EPO is as follows:

| | |
|---|---|
| ATGGGCGTGC CCGAGCGCCC CACCCTGCTG CTGCTGCTGA GCCTGCTCCT GATCCCCCTG | (SEQ ID NO:1) |
| GGCCTGCCCG TGCTGTGCGC CCCCCCCCGC CTGATCTGCG ACAGCCGCGT GCTGGAGCGC | |
| TACATCCTGG AGGCCAAGGA GGCCGAGAAC GTGACCATGG GCTGCGCTGA GGGCCCCCGC | |
| CTGAGCGAGA ACATCACCGT GCCCGACACC AAGGTGAACT TCTACGCCTG GAAGCGCATG | |
| GAGGTGGAGG AGCAGGCCAT CGAGGTGTGG CAGGGCCTGT CCCTGCTGTC TGAGGCCATC | |
| CTGCAGGCCC AGGCCCTGCT GGCCAACTCC TCCCAGCCCC CCGAGACCCT GCAGCTGCAC | |
| ATCGACAAGG CCATCAGCGG CCTGCGCTCC CTGACCTCCC TGCTGCGCGT GCTGGGCGCC | |
| CAGAAGGAGC TGATGAGCCC CCCCGACACC ACCCCCCCCG CCCCCCTGCG CACCCTGACC | |
| GTGGACACCT TCTGCAAGCT GTTCCGCGTG TACGCCAACT TCCTGCGCGG CAAGCTGAAG | |
| CTGTACACCG GCGAGGTGTG CCGCCGCGGC GACCGCTGA. | |

Plasmid DNA was prepared by standard double CsCl gradient purification and resuspended in sterile saline solution.

Animals and treatment—Six week old female Balb/C mice and 10 week old female rabbits were purchased from Charles River Breeding Laboratory and used in all experiments. Animals were maintained in standard conditions under 12-hr light-dark cycle, provided irradiated food and chlorinated water ad libitum. All animal procedures were conducted in conformity with national and international laws and policies.

Electro-gene transfer—Mouse quadriceps and rabbit tibialis anterior muscle were surgically exposed and injected with a predetermined amount of plasmid DNA. The volume of injection was kept constant at 50 μl in mice and 200 μl in rabbits. Where indicated, hyaluronidase was resuspended in 50 μl (mice) or 500 μl (rabbits) of sterile saline solution at the desired concentration and injected prior to EGT at the indicated time. HYAse may be purified as disclosed by Borders, et al., 1965, J. Biol. Chem. 243: 3750-3762. For these studies, HYAse was obtained from Sigma (St. Louis. MO); Type: VI-S from Bovine Testes; Enzyme Commission Number: 3.2.1.35; Synonyms: Hyaluronoglucosamidase, Hyaluronate 4-glycanohydrolase; Source: Bovine testes). Steel electrodes in the form of parallel 0.2 mrn wires about 3 cm long and 5 mm apart were inserted intramuscularly around the injection site. The electric field was applied in a pulsed form as described (Rizzuto et al., 1999, Proc. Natl. Acad. Sci. 96:6417-6422) with minor modifications. Briefly, mouse quadriceps muscles were surgically exposed and injected with a predetermined amount of plasmid DNA. Steel electrodes in the form of parallel 0.2 mm wires about 3 cm long and 5 mm apart were brought into contact with the muscle in parallel orientation with respect to the muscle fibres. The electric field was applied in a pulsed form through a Pulsar 6 bp-a/s bipolar stimulator (FHC, ME, USA) and each cycle of stimulation comprised a one second pulse train of square bipolar pulses delivered every other second. Each train consisted in 10³ pulses of 200 μsec length and 45 Volts amplitude. Pulses were monitored using a digital oscilloscope. A custom amplifier was constructed using an APEX PA-85 power operational amplifier in the output stage (APEX Technologies, Tucson, Ariz.). Signals were generated by an integrated custom signal generator and were monitored using a two-channel 8-bit oscilloscope card (K7103 Velleman, Gavere, Belgium). The entire set up was controlled by a custom software package written in Java programming language running on PC-compatible laptop (Extensa 501T, Acer America, San Jose, Calif.). Voltage and current were measured periodically during the experiment with a digital oscilloscope. Voltage was monitored across the lower resistor of a voltage divider (100,000 ohms resistor over a 10,000 ohm resistor) in parallel with the electrodes, whereas current was monitored by measuring the potential drop across a precision 1 ohm resistor in series with the electrodes.

Histological analysis—Mouse quadriceps and rabbit tibialis were removed at the indicated time after treatment and fixed 3 h in ice in 0.50% glutaraldehyde and 2% paraformaldehyde in sodium phosphate buffer (pH 7.4) containing 0.02% Nonidet P-40. After three washes in ice cold PBS muscles were incubated in a reaction mixture containing 2 mM 5-bromo-4 chloro-3-indolyl-β—D-galactosidase (X-gal, GIBCO BRL), 2 mM MgCl₂, 4 mM potassium ferricyanide, 4 mM potassium ferrocyanide, 0.02% Nonidet P40 in sodium phosphate buffer at 30° C. overnight. After incubation, quadriceps were washed three times in PBS and embedded in 20% sucrose in sodium phosphate buffer (pH 7.4) for 5 hrs at 4° C. Cryostatic muscle sections were finally examined for β-galactosidase expression by light microscopy. For the assessment of tissue damage and morphology, tissues were embedded in paraffin, stained with Haematoxylin-Eosin (H/E) and examined under light microscope as described (Ausubel et al., 1992).

Hematocrit, EPO, and SeAP measurement—Blood samples were collected at the indicated times. Hematocrits were determined by centrifugation of whole blood in heparinized capillary tubes as previously described (Rizzuto et al., 1999, Proc. Natl. Acad. Sci. 96:6417-6422). Serum SeAP levels were monitored by Phospha-Light (Chemiluminescent Reporter Assay for secreted alkaline phosphatase), Tropix. Results were analyzed by using ANOVA analysis (STAT-VIEW, Abacus Concept Berkeley Calif.). A P value <0.05 was considered significant.

Results—Effect of Hyaluronidase on EPO gene expression in mouse muscle. To assess the effect of HYAse on EPO gene expression, the quadriceps muscle of groups of 4 Balb/c mice were injected with different amounts of HYAse ranging from 0.5 to 90 U. Four hours later, 3 μg of plasmid pCMV/mEPO were injected in the same muscle and the treated tissue was subjected to ES as previously described above. EPO levels and Hct values of treated mice were determined one week after injection and compared to those of a control group that did not receive HYAse and was injected with the same amount of pCMV/mEPO. As shown in FIG. 1A, increase of EPO values in DNA injected and muscle ES mice was significant over that of saline treated animals (121.8 mU/ml) (1 mU=10 pg). Pre injection of 90 U of HYAse resulted in a five-fold increase in EPO expression (550 mU/ml). Similarly, pre injection of 36 U of HYAse also resulted in a considerable increase in EPO levels, albeit slightly lower than that observed with 90 U (455 mU/ml). In contrast, injection of 18, 5.4, 1.8, or 0.5 U of HYAse did not result in a substantial increase in EPO levels as compared to injection of plasmid DNA alone. No increase in EPO levels was observed upon injection of HYAse and up to 100 μg of plasmid pCMV/mEPO in mice that were not subjected to ES.

The increase in serum EPO levels resulted in a notable increment in the Hct of treated animals as compared to saline treated controls (FIG. 1B). However, at this time point, the measured Hct values did not significantly differ among the different treated groups. The lack of a quantitative difference between the Hct values of the various mice groups at this time point simply reflects the notion that erythropoiesis is regulated by a series of factors in addition to EPO that can limit the progression of Hct increase. Nonetheless, these results indicate that pre-injection of HYAse leads to an increase in EPO gene expression upon DNA injection and muscle ES.

To determine the pre-injection time for HYAse administration required for maximal EPO gene expression, groups of Balb/c mice were injected with HYAse 4 h, 1 h, and 10 minutes prior to EGT. The amount of HYAse injected into the mouse quadriceps was fixed at 36 U, since it corresponds to 720 U/ml and is within the range of HYAse used for clinical applications (150 to 1500 U; Berger, 1984, J. Am. Geriatr. Soc.32: 199-203). As shown in FIG. 2, measurement of EPO levels one-week post injection (p.i.) indicated that HYAse injection 4 or 1 hr and 10 min prior to DNA injection and muscle ES resulted in 5-fold increase in serum EPO level as compared to that of mice injected with DNA alone. Thus, these data demonstrate that administration of HYAse 10 min prior to electroinjection of plasmid is sufficient to lead to a significant increase in EPO gene expression in mice.

Long term effect of HYAse injection on EPO gene expression—Verification as to whether the increase of EPO gene expression upon HYAse administration could be observed over time and independently of DNA dosage was undertaken. To this end, groups of animals were injected with different amounts of plasmid pCMV/mEPO and serum EPO levels of mice that had been pre injected with HYAse were measured over time and compared to those of mice treated with DNA alone (FIG. 3A-C). Serum EPO levels measured at 7, 56, and 120 days p.i. correlated with the amount of injected DNA. The EPO values observed in mice that had been pretreated with HYAse were consistently higher than those of animals treated with DNA alone and ranged from 64 mU/ml with 0.5 μg of DNA to 1324 mU/ml at day 7 in mice injected with 50 μg of DNA. Groups pretreated with HYAse and injected with 3, 10, 50 μg of plasmid DNA showed EPO levels that were significantly different from those detected in mice electroinjected with DNA alone. In all treated groups, circulating EPO reached a peak level at 7 days p.i. (FIG. 3A), decreased variably in the different groups to from ½ to ⅛ of the initial value after 56 days (FIG. 3B), and remained constant thereafter. Additionally, because of the high level of EPO expression, mice injected with 50 μg of plasmid DNA after HYAse administration displayed extremely high Hct values (>85%) and died by 120 days p.i. (FIG. 3C). These results demonstrate that injection of HYAse results in enhanced EPO expression independently of DNA dosage and that such effect persists for a prolonged period of time.

Analysis of tissue damage—The extent of tissue alterations that could be associated to the use of HYAse for EPO gene transfer was assessed. Histology analysis of HYAse injected quadriceps muscles was performed 1, 3, 7, and 30 days p.i. No tissue alteration was detected 24 hrs after injection. The most striking and consistent pathological findings were observed only in samples 3 days after the treatment (FIG. 4A). In these samples lesions were detected in about the 20% of the total muscle mass. In paraffin embedded sections stained with H/E, areas of massive colliquative necrosis of the muscle fibers were observed, in each necrotic area mononuclear cell infiltrates, mostly of macrophagic origin, were detected. Each area was typically surrounded by a reactive fibrosis. Similar and consistent necrotic lesions were observed also in samples at 7 days after treatment but in this case they represented roughly 1% the total muscle mass (FIG. 4B). After 7 days no fibrotic reaction was observed and mononuclear cells infiltrate were less apparent. The necrotic lesions after 1 month were sporadic and less than 1% of the muscle mass was involved. Additionally, mononuclear cell infiltrates were no longer detected (FIG. 4C). Therefore, these findings suggest that HYAse treatment results in limited and transient tissue damage.

Effect of HYAse on gene transfer in large muscles—To assess the effect of HYAse administration on EPO gene expression of large muscles, a series of DNA injection experiments were carried out on rabbit tibialis anterior muscle. Construct pCMV/mEPOopt was utilized for these studies. This plasmid carries a mouse EPO cDNA codon-optimized to mammals. Codon-optimized EPO constructs have been reported to express higher EPO levels (Kim et al., 1997, Gene 199: 293-301). Additionally, to verify that the effects of HYAse were not limited to EPO gene expression, rabbits were injected with a plasmid encoding secreted alkaline phosphatase (pCMV/SeAP) (Bettan et al, 1994, Anal. Biochem. 271: 187-189). As shown in FIG. 5A-B, treatment with 180 U of HYAse 40 min prior to injection of 200 μg of pCMV/mEPOopt and ES resulted into a 3 fold increase in EPO expression (FIG. 5A). The amount of HYAse injected and time of injection were those that yielded the greater level of EPO expression. A 17-fold increase in expression was detected in HYAse treated rabbits upon injection of 200 μg of pCMV/SEAP and muscle ES (FIG. 5B). The same increment of SEAP expression was also noted on injection of 0.5 and 1 mg of plasmid DNA. These results confirm the effect of HYAse on gene expression observed in mice and demonstrate that efficiency of gene expression is increased upon DNA injection and muscle ES in large animals.

Effect of HYAse on β-galactosidase gene expression in muscle—To analyze the effects of HYAse on tissue distribution of gene expression following EGT of plasmid DNA, the β-galactosidase gene was utilized. Two hundred micrograms of plasmid pCMV/β-gal/NLS encoding the E. coli lacZ fused to a nuclear localization signal were injected into the tibialis muscle 40 mins after HYAse administration. The treated muscles were subjected to EGT. Additionally, the extent of β-gal expression was compared to that of rabbits treated with EGT alone. The histology analysis demonstrated that the area of positive X-gal staining was significantly larger in rabbits pretreated with HYAse than those in animals treated with DNA alone (FIG. 6). These results indicate that HYAse promotes distribution of plasmid DNA across the tissue.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of delivering a pharmaceutical agent into a tissue of a vertebrate host, which comprises:
a) administering a biologically effective amount of hyaluronidase to the tissue of the vertebrate host;
b) administering the pharmaceutical agent proximal to the delivery site of hyaluronidase administration of step a); and,
c) applying an electrical stimulus proximal to the delivery points of step a) and step b), such that the amount of biologically effective pharmaceutical agent delivered to the tissue of the vertebrate host is greater than application of an electrical stimulus and pharmaceutical agent alone.

2. A method of claim 1 wherein the vertebrate host is a mammalian host.

3. A method of claim 2 wherein the mammalian host is a non-human primate.

4. A method of claim 2 wherein the mammalian host is a human.

5. A method of claim 4 wherein human muscle tissue of the human is targeted for delivery of the pharmaceutical agent.

6. A method of claim 5 wherein the human muscle tissue is skeletal muscle tissue.

7. A method of claims 1, 2, 3, 4, 5, or 6 wherein the pharmaceutical agent is a nucleic acid molecule.

8. The method of claim 7 wherein the nucleic acid molecule is a DNA plasmid molecule.

9. A method of delivering a pharmaceutical agent into a tissue of a vertebrate host, which comprises:
a) administering a biologically effective amount of hyaluronidase to the tissue of the vertebrate host up to about 4 hours prior to application of an electrical stimulus;
b) administering the pharmaceutical agent proximal to the delivery site of hyaluronidase administration of step a); and,
c) applying the electrical stimulus proximal to the delivery points of step a) and step b), such that the amount of biologically effective pharmaceutical agent delivered to the tissue of the vertebrate host is greater than application of an electrical stimulus and pharmaceutical agent alone.

10. A method of claim 9 wherein the vertebrate host is a mammalian host.

11. A method of claim 10 wherein the mammalian host is a non-human primate.

12. A method of claim 10 wherein the mammalian host is a human.

13. A method of claim 12 wherein human muscle tissue of the human is targeted for delivery of the pharmaceutical agent.

14. A method of claim 13 wherein the human muscle tissue is skeletal muscle tissue.

15. A method of claims 9, 10, 11, 12, 13, 14 or 15 wherein the pharmaceutical agent is a nucleic acid molecule.

16. The method of claim 15 wherein the nucleic acid molecule is a DNA plasmid molecule.

17. A method of delivering a pharmaceutical agent into a tissue of a vertebrate host, which comprises:
a) administering a biologically effective amount of hyaluronidase to the tissue of the vertebrate host up to about 4 hours prior to application of an electrical stimulus;
b). administering the pharmaceutical agent proximal to the delivery siture of hyaluronidase administration of step a); and,
c) applying the electrical stimulus proximal to the delivery points of step a) and step b), such that the amount of biologically effective pharmaceutical agent delivered to the tissue of the vertebrate host is greater than application of an electrical stimulus and pharmaceutical agent alone, wherein the hyaluronidase of step a) and the pharmaceutical agent of step b) comprise a single formulation, said formulations being administered prior to or in conjunction with the application of the electrical stimulus of step c).

18. A method of claim 17 wherein the vertebrate host is a mammalian host.

19. A method of claim 18 wherein the mammalian host is a non-human primate.

20. A method of claim 18 wherein the mammalian host is a human.

21. A method of claim 20 wherein human muscle tissue of the human is targeted for delivery of the pharmaceutical agent.

22. A method of claim 21 wherein the human muscle tissue is skeletal muscle tissue.

23. A method of claims 17, 18, 19, 20, 21, or 22 wherein the pharmaceutical agent is a nucleic acid molecule.

24. The method of claim 23 wherein the nucleic acid molecule is a DNA plasmid molecule.

25. A method of claims 1, 9 or 17 wherein hyaluronidase is administered by direct needle injection.

26. A method of claim 1 wherein the hyaluronidase of step a) is added from about 30 minutes to 2 hours to application of the electrical stimulus of step c).

27. A method of claim 1 wherein the hyaluronidase of step a) is added from about 15 minutes to 45 minutes to application of the electrical stimulus of step c).

28. A formulation which comprises hyaluronidase and a pharmaceutical agent.

29. A formulation of claim 28 wherein the pharmaceutical agent is a nucleic acid molecule.

30. A formulation of claim 29 wherein the nucleic acid molecule is a DNA plasmid molecule.
